(19) United States
(12) Patent Application Publication
(10) Pub. No.: US 2016/0139022 A1
(43) Pub. Date: May 19, 2016

(54) Device of testing particles in tail gas exhausted from automobile

(71) Applicant: Li Wan Yin, N.T. (HK)

(72) Inventor: Li Wan Yin, N.T. (HK)

(21) Appl. No.: 14/547,143

(22) Filed: Nov. 19, 2014

(51) Int. Cl. G01N 15/06; G01N 33/00

(52) U.S. Cl. CPC G01N 15/0618; G01N 33/0036

(57) ABSTRACT

A device of testing particles in a tail gas exhausted from an automobile contains: an intake unit, a smoke detection unit, a processing unit, and a filtering unit. The filtering unit includes a filtration chamber, a filter paper, and two weighing elements. The filtration chamber has a locking portion and an opening. A transmission belt is mounted beside two sides of the filtering unit and has an upstream segment and a downstream segment, and between the upstream segment of the transmission belt and the locking portion is defined a first weighing element, between the locking element and the downstream segment of the transmission belt is defined a second weighing element. A first delivery sucker, a second delivery sucker, and a third delivery sucker are all mounted in a filtration chamber. Thereby, a total weight of particles in a tail gas is measured within a unit time.

### FIELD OF THE INVENTION

The present invention relates to a device of testing particles in a tail gas exhausted from an automobile which measures a total weight of particles in a tail gas accurately within a unit time.

### BACKGROUND OF THE INVENTION

A conventional testing device is employed to test gas (such as gaseous nitrogen oxides or carbon monoxide) and solid particles (such as carbon black particles) exhausted from an automobile.

The solid particles in the tail gas are filtered by a filter paper and tested by the conventional testing device. The conventional testing device tests black smokes (i.e., carbon black particles) on the filter paper by which smokes are calculated. However, a total weight of the solid particles in the tail gas cannot be measured within a unit time.

The present invention has arisen to mitigate and/or obviate the afore-described disadvantages.

### SUMMARY OF THE INVENTION

The primary object of the present invention is to provide a device of testing particles in a tail gas exhausted from an automobile which measures a total weight of particles in a tail gas accurately within a unit time.

A device of testing particles in a tail gas exhausted from an automobile provided by the present invention contains: an intake unit, a smoke detection unit, and a processing unit.

A filtering unit is fixed between the intake unit and the processing unit, the filtering unit includes a filtration chamber communicating with the intake unit and the processing unit, a filter paper, two weighing elements under the filter paper, the filtration chamber has a locking portion and an opening defined on a middle portion thereof to flow a tail gas, the filter paper is covered on the opening.

A transmission belt is mounted beside two sides of the filtering unit and has an upstream segment coupled with the filtration chamber and a downstream segment joined with the smoke detection unit, and between the upstream segment of the transmission belt and the locking portion is defined a first weighing element, between the locking element and the downstream segment of the transmission belt is defined a second weighing element, the first weighing element weighs the filter paper before a filtration process, and the second weighing element weighs the filter paper after the filtration process.

A first delivery sucker, a second delivery sucker, and a third delivery sucker are all mounted in a filtration chamber, wherein the first delivery sucker delivers a filter paper from the first weighing element to the locking portion, the second delivery sucker delivers the filter paper from the locking portion to the second weighing element, and the third delivery sucker delivers the filter paper from the second weighing element to the downstream segment of the transmission belt.

Preferably, a first conveying roller and a second conveying roller are located at two sides of a lower end of the opening, and the first conveying roller conveys the filter paper toward the locking portion, the second conveying roller conveys the filter paper away from the opening after the filtration process.

### BRIEF DESCRIPTION OF THE DRAWINGS

FIG. 1 is a side plane view showing the assembly of a device of testing particles in a tail gas exhausted from an automobile according to a first embodiment of the present invention.

FIG. 2 is a side plan view the assembly of a locking portion and an opening of the device of testing particles in a tail gas exhausted from an automobile according to a first embodiment of the present invention.

FIG. 3 is a side plane view showing the assembly of a device of testing particles in a tail gas exhausted from an automobile according to a second embodiment of the present invention.

### DETAILED DESCRIPTION OF THE PREFERRED

### Embodiments

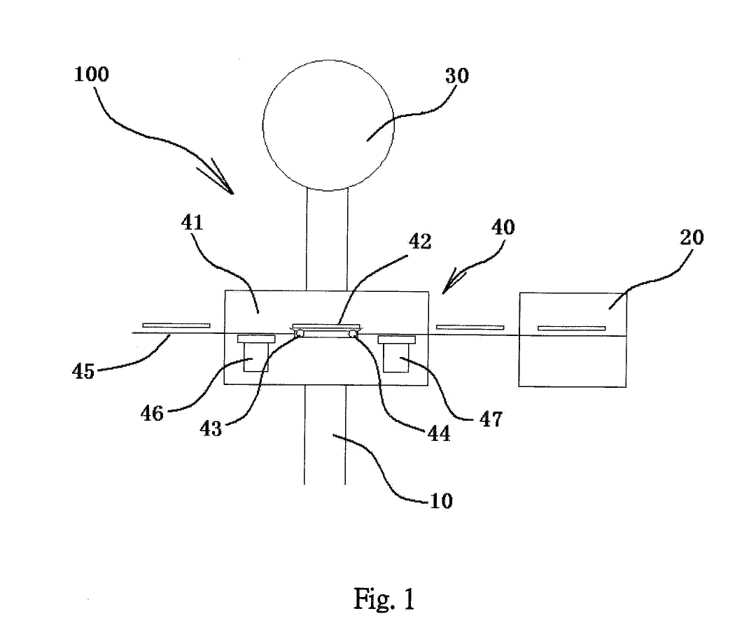

With reference to FIG. 1, a device 100 of testing particles in a tail gas exhausted from an automobile according to a first embodiment of the present invention comprises: an intake unit 10, a smoke detection unit 20, a processing unit 30, a filtering unit 40 fixed between the intake unit 10 and the processing unit 30. The filtering unit 40 includes a filtration chamber 41 communicating with the intake unit 10 and the processing unit 30, a filter paper 42, two weighing elements under the filter paper 42 to weigh the filter paper 42 after and before a filtration process. The filtration chamber 41 of the filtering unit 40 has the filter paper 42 disposed therein to filter a tail gas flowing through the filtration chamber 41. The filtration chamber 41 also has a locking portion 411 and an opening 412 defined on a middle portion thereof to flow the tail gas, wherein the filter paper 42 is covered on the opening 412.

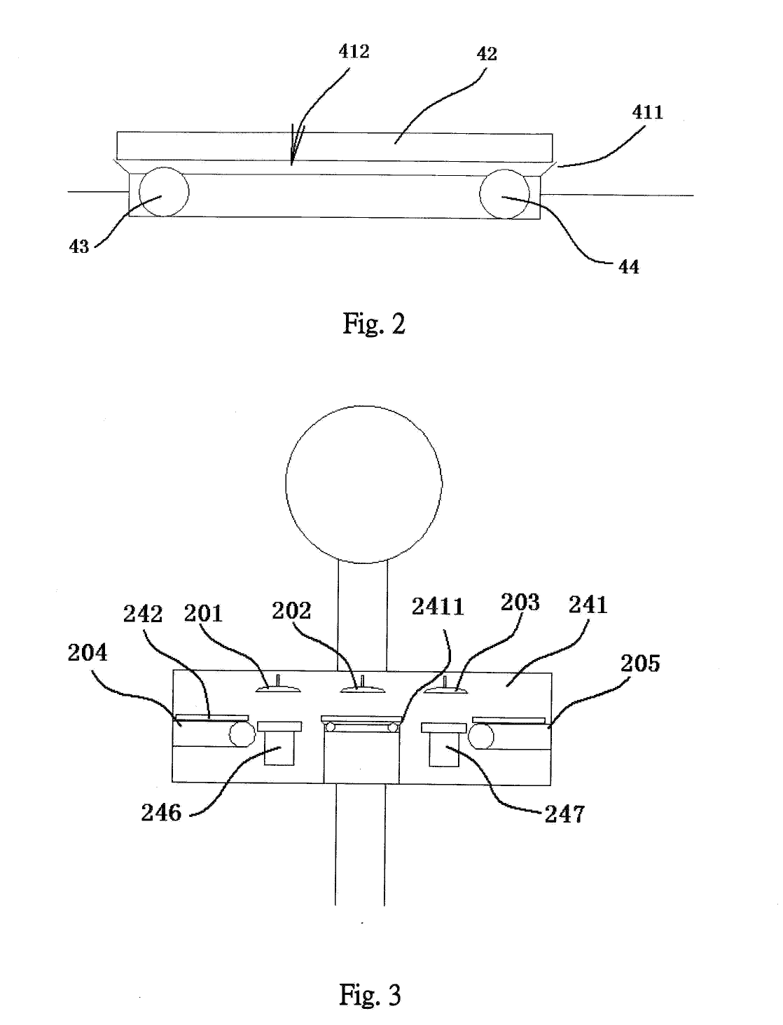

Referring to FIG. 2, a first conveying roller 43 and a second conveying roller 44 are located at two sides of a lower end of the opening 412, wherein the first conveying roller 43 conveys the filter paper 42 toward the locking portion 411, and the second conveying roller 44 conveys the filter paper 42 away from the opening 412. In other words, the first conveying roller 43 and the second conveying roller 44 convey the filter paper 42 in the filtration chamber 41 based on a setting time to replace the filter paper 42.

Furthermore, a transmission belt 45 is mounted beside two sides of the filtering unit 40 and has an upstream segment coupled with the filtration chamber 41 and a downstream segment joined with the smoke detection unit 20.

Between the upstream segment of the transmission belt 45 and the first conveying roller 43 is defined a first weighing element 46, and between the second conveying roller 44 and the downstream segment of the transmission belt 45 is defined a second weighing element 47. The first weighing element 46 weighs the filter paper 42 before the filtration process, and the second weighing element 47 weighs the filter paper 42 after the filtration process, such that a total weight of particles in the tail gas is measured within a unit time.

As shown in FIG. 3, a difference of a device 100 of testing particles in a tail gas exhausted from an automobile of a second embodiment from that of the first embodiment comprises: a first delivery sucker 201, a second delivery sucker 202, a third delivery sucker 203 which are all mounted in a filtration chamber 241, wherein the first delivery sucker 201 delivers a filter paper 242 from the first weighing element 246 to a locking portion 2411, the second delivery sucker 202 delivers the filter paper 242 from the locking portion 2411 to the second weighing element 247, and the third delivery sucker 203 delivers the filter paper 242 from the second weighing element 247 to a downstream segment 205 of a transmission belt. Furthermore, an upstream segment 204 of the transmission belt moves the filter paper 242 to the first weighing element 246, and the first delivery sucker 201, the second delivery sucker 202 and the third delivery sucker 203 deliver the filter paper 242 in the filtration chamber 241.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A device of testing particles in a tail gas exhausted from an automobile comprising: an intake unit, a smoke detection unit, and a processing unit; characterized in that:
a filtering unit is fixed between the intake unit and the processing unit, the filtering unit includes a filtration chamber communicating with the intake unit and the processing unit, a filter paper, two weighing elements under the filter paper, the filtration chamber has a locking portion and an opening defined on a middle portion thereof to flow a tail gas, the filter paper is covered on the opening;
a transmission belt is mounted beside two sides of the filtering unit and has an upstream segment coupled with the filtration chamber and a downstream segment joined with the smoke detection unit, and between the upstream segment of the transmission belt and the locking portion is defined a first weighing element, between the locking element and the downstream segment of the transmission belt is defined a second weighing element, the first weighing element weighs the filter paper before a filtration process, and the second weighing element weighs the filter paper after the filtration process;
a first delivery sucker, a second delivery sucker, and a third delivery sucker are all mounted in a filtration chamber, wherein the first delivery sucker delivers a filter paper from the first weighing element to the locking portion, the second delivery sucker delivers the filter paper from the locking portion to the second weighing element, and the third delivery sucker delivers the filter paper from the second weighing element to the downstream segment of the transmission belt.

2. The device of testing the particles in the tail gas exhausted from the automobile as claimed in claim 1, wherein a first conveying roller and a second conveying roller are located at two sides of a lower end of the opening, and the first conveying roller conveys the filter paper toward the locking portion, the second conveying roller conveys the filter paper away from the opening after the filtration process.
